(19) United States
(12) Patent Application Publication
(10) Pub. No.: US 2026/0248994 A1
(43) Pub. Date: Aug. 27, 2026

(54) STEERABLE SUCTION/IRRIGATION SYSTEM FOR SINGLE-PORT OPERATION

(71) Applicants: Jaschar SHAKURI-RAD, Morgantown, WV (US); Justin R. CHAMBERS, Morgantown, WV (US); Ryan GROSS, Morgantown, WV (US)

(72) Inventors: Jaschar SHAKURI-RAD, Morgantown, WV (US); Justin R. CHAMBERS, Morgantown, WV (US); Ryan GROSS, Morgantown, WV (US)

(21) Appl. No.: 19/552,463

(22) Filed: Feb. 27, 2026

Related U.S. Application Data
(60) Provisional application No. 63/763,980, filed on Feb. 27, 2025.
(60) Provisional application No. 63/886,967, filed on Sep. 24, 2025.

(51) Int. Cl. A61M 1/00; A61M 13/00; A61M 25/01

(52) U.S. Cl. CPC A61M 1/77; A61M 1/7413; A61M 13/003; A61M 25/0133

(57) ABSTRACT

A medical device includes: a cannula having a distal end and a proximal end; a housing connected to the proximal end; and a manipulator connected to the housing; wherein the cannula includes a rigid component that extends from the proximal end to an intermediate location between the proximal end and the distal end; the cannula comprises a flexible component that extends from the proximal end to the distal end; the cannula includes a rigid section in which the flexible component is inside the rigid component and a flexible section in which the flexible component is outside the rigid component; the manipulator is connected to the flexible component in a manner that permits selective bending of the flexible section relative to the rigid section in response to user input at the manipulator; and the flexible component includes a channel that is configured to convey suction and/or irrigation at the distal end.

### PRIORITY

This application claims priority to U.S. provisional patent applications number 63/763,980 filed Feb. 27, 2025, and 63/886,967 filed Sep. 24, 2025, the contents of both of which are incorporated by reference herein in their entirety.

### FIELD OF THE INVENTION

Aspects of the present invention relate generally to single-port surgery and, more particularly, to devices and methods for providing a steerable suction/irrigation for use in single-port surgery.

### BACKGROUND

Single-port surgery is a procedure performed through one cut (i.e., incision) and with an innovative single-port robot. Surgeons perform single-port surgery through a single port (i.e., incision) in the patient's body, such as in the navel or abdomen. The location of the single incision may vary depending on the surgical procedure. First used clinically in September 2018, single-port robotic surgery is now widely used in many medical centers worldwide for various procedures. Surgeons use this minimally invasive approach in many procedures involving different organ systems. For example, surgeons in a variety of specialties conduct single-port surgery, including urology, and ear, nose and throat. Because it uses only one incision, single-port surgery leaves little to no scarring and may reduce complications that commonly occur after traditional open and even traditional laparoscopic abdominal surgery. People who have single-port surgery report less pain and faster recovery than with traditional minimally invasive surgeries. However, the single-port approach is more challenging than traditional laparoscopy or robotic surgery because your surgeon has less freedom of movement with all instruments using the same entry point.

Current laparoscopic handheld suction/irrigation devices are cumbersome to use and are difficult to maneuver, especially during single-port robotic surgery. Because single-port robotic systems rely on a singular port configuration to reduce the need for additional incisions and ports, this creates space limitations that current tools are not designed to accommodate.

Current tools are also designed with singular functionality and are focused on the suctioning task. In order to manipulate tissue, the suction device has to be taken out of the port and a new tool inserted. This limits the bedside surgeon and assistants.

### SUMMARY

Implementations of the invention address the above-noted problems of the prior art by providing an inventive suction/irrigation device that allows bedside assistants and surgeons to overcome the space limitations faced during laparoscopic/robotic surgery and allow for more efficient surgical procedures. The inventive device allows more efficient and space conscious suctioning, while allowing manipulation of tissue, and allowing specimen retrieval through the port, amongst others. Embodiments described herein include a suction/irrigation device preferably used in single-port laparoscopic operations. A preferred embodiment comprises a suction component, an irrigation component, a cannula, and a cannula tip that is designed to be steerable or manipulable to access hard to reach areas and offer better overall control and manipulation during procedures. The preferred embodiment includes a medical device comprising: a cannula having a distal end and a proximal end opposite the distal end; a housing connected to the proximal end of the cannula; and a manipulator connected to the housing; wherein the cannula includes a rigid component that extends from the proximal end of the cannula to an intermediate location between the proximal end of the cannula and the distal end of the cannula; the cannula comprises a flexible component that extends from the proximal end of the cannula to the distal end of the cannula; the cannula includes a rigid section in which the flexible component is inside the rigid component and a flexible section in which the flexible component is outside the rigid component; the manipulator is connected to the flexible component in a manner that permits selective bending of the flexible section relative to the rigid section in response to user input at the manipulator; and the flexible component includes a channel that is configured to convey suction and/or irrigation at the distal end of the cannula.

### BRIEF DESCRIPTION OF THE SEVERAL VIEWS OF THE DRAWINGS

Aspects of the present invention are described in the detailed description which follows, in reference to the noted plurality of drawings by way of non-limiting examples of exemplary embodiments of the present invention.

FIG. 1 shows prior art suction/irrigation device used in single-port robotic surgery.

FIG. 2A shows a suction/irrigation device used in single-port robotic surgery in accordance with aspects of the present disclosure.

FIG. 2B shows a configuration of a cannula that may be used with a suction/irrigation device in accordance with aspects of the present disclosure.

FIG. 2C shows a configuration of a cannula that may be used with a suction/irrigation device in accordance with aspects of the present disclosure.

FIG. 2D shows a configuration of a housing that may be used with a cannula in accordance with aspects of the present disclosure.

FIGS. 3A and 3B show aspects of a suction/irrigation device usable in single-port robotic surgery in accordance with aspects of the present disclosure.

FIGS. 4A and 4B show aspects of a suction/irrigation device usable in single-port robotic surgery in accordance with aspects of the present disclosure.

FIG. 5A-5D show aspects of a suction/irrigation device usable in single-port robotic surgery in accordance with aspects of the present disclosure.

FIG. 6A-6D show aspects of a suction/irrigation device usable in single-port robotic surgery in accordance with aspects of the present disclosure.

FIG. 7A-7C show aspects of a suction/irrigation device usable in single-port robotic surgery in accordance with aspects of the present disclosure.

FIG. 8 shows a configuration of a grasping actuator that may be used with a cannula in accordance with aspects of the present disclosure.

FIG. 9 shows a configuration of a grasping actuator that may be used with a cannula in accordance with aspects of the present disclosure.

FIG. 10 shows aspects of a suction/irrigation device usable in single-port robotic surgery in accordance with aspects of the present disclosure.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details in more detail than is necessary for the fundamental understanding of aspects of the present invention, the description taken with the drawings making apparent to those skilled in the art how several forms of the present invention may be embodied in practice.

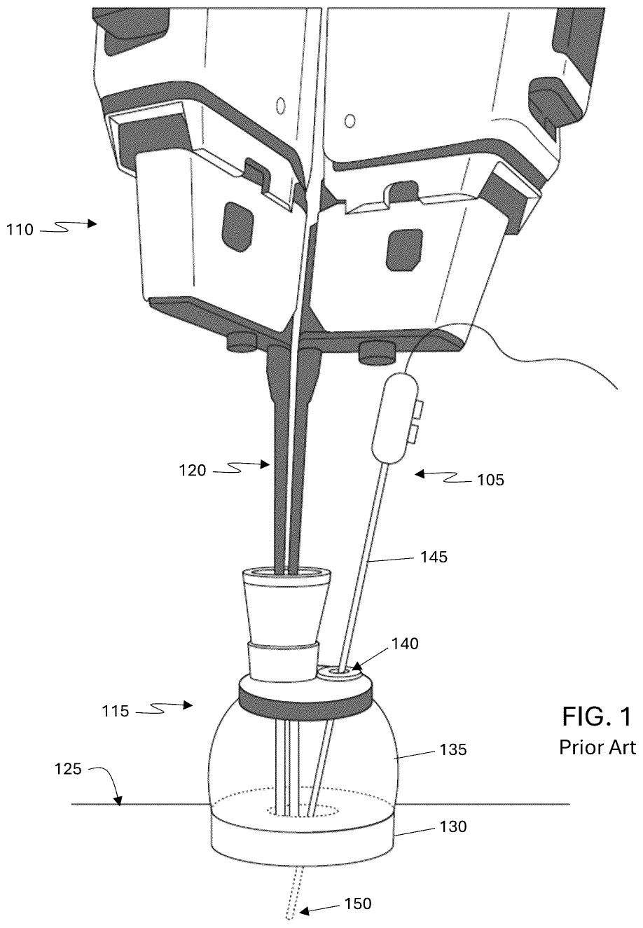

FIG. 1 shows prior art suction/irrigation device 105 used in single-port robotic surgery. In the example shown in FIG. 1, the suction/irrigation device 105 is used with a robotic surgery system such as the DA VINCI SP, which includes a robotic system 110 and an access kit 115. (DA VINCI SP is a trademark of Intuitive Surgical Operations, Inc. of Sunnyvale, California.) The robotic system 110 includes one or more robotic instruments 120 that enter the patient through a single port (i.e., incision) in the skin 125 of the patient. The robotic instruments 120 may include, for example, an endoscope, scissors, cautery instrument, forceps, needle driver, retractor, and clip applier. A surgeon controls movement of the robotic instruments 120 via hand inputs to the robotic system 110. The access kit 115 directly contacts the skin 125 of the patient around the incision, and the robotic instruments 120 access the incision by passing through one or more ports in the access kit 115. The access kit 115 may include a wound protector 130, as well as a clear spherical attachment 135 (also known as a “fishbowl”) which has three separate ports including one for a trocar, one for 12 mm instrumentation, and one for an 8 mm assistant port 140 on the side of the sphere, for example.

With continued reference to FIG. 1, the suction/irrigation device 105 may be used to access the incision via the assistant port 140. The suction/irrigation device 105 includes a rigid tube 145 with a distal end 150, the tube 145 typically constructed of rigid metallic tubing. An operator such as a surgeon or assistant may physically manipulate the position of the suction/irrigation device 105 to attempt to locate the distal end 150 of the suction/irrigation device 105 at a desired location inside the patient. However, when manipulating the position of the suction/irrigation device 105, the suction/irrigation device 105 may come into contact with the structure of the robotic system 110 thereby blocking the ability to locate the distal end of the suction/irrigation device 105 at the desired location inside the patient. Moreover, the straight and rigid tube 145 of the suction/irrigation device 105 combined with the limited range of motion allowed by the combination of the assistant port 140 and incision further makes it difficult to locate the distal end 150 of the suction/irrigation device 105 at the desired location inside the patient. The rigid nature of the tube 145 combined with these space constraints makes it difficult to locate the distal end of the suction/irrigation device 105 at the desired location inside the patient.

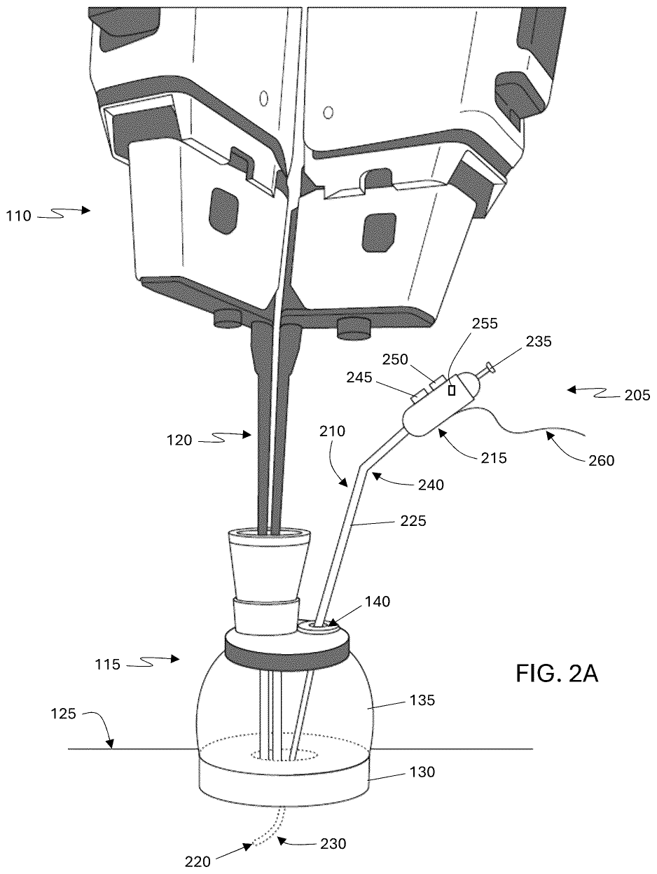

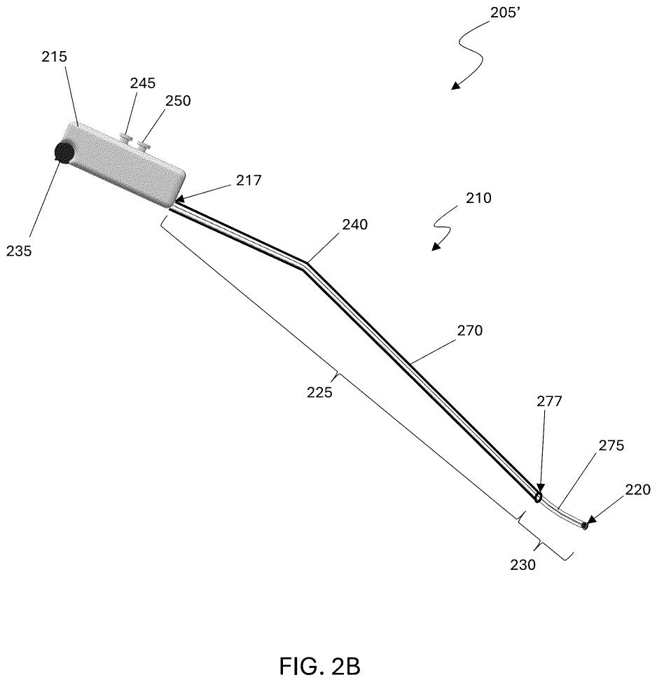

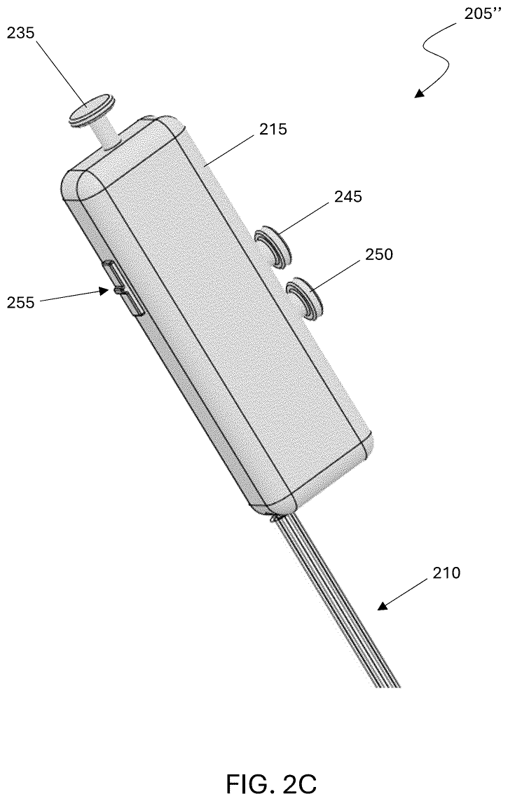

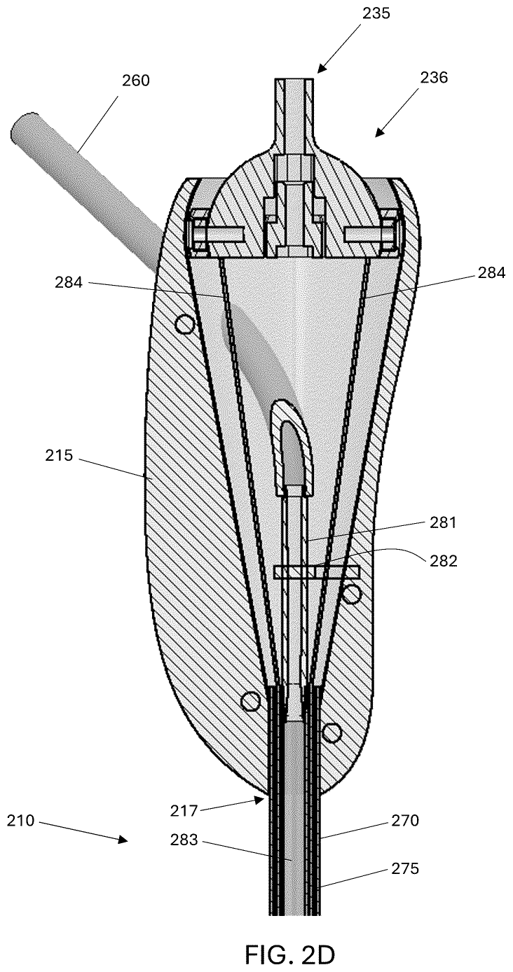

FIG. 2A shows a suction/irrigation device 205 used in single-port robotic surgery in accordance with aspects of the present disclosure. As shown in FIG. 2A, the suction/irrigation device 205 may be used with the robotic system 110 of FIG. 1, which includes one or more robotic instruments 120 that access a single incision in a patient skin 125 via an access kit 115. As in FIG. 1, the access kit 115 may include a wound protector 130 and a clear spherical attachment 135, and the robotic instruments 120 and the suction/irrigation device 205 may access the incision via one or more ports (e.g., assistant port 140) formed in the access kit 115.

With continued reference to FIG. 2A, in various embodiments the suction/irrigation device 205 includes a cannula 210 and a housing 215. In embodiments, the cannula 210 includes a distal end 220 that is configured to be inserted into the patient, e.g., via the access kit 115 or other type of wound covering device used in single-port surgery. In embodiments, the housing 215 is at a proximal end 217 of the cannula 210 opposite the distal end 220. In accordance with aspects of the present disclosure, the cannula 210 includes a rigid section 225 adjacent to the housing 215 and a flexible section 230 adjacent to the distal end 220. In embodiments and as described herein, the flexible section 230 is manipulatable by an operator via a manipulator 235 of the suction/irrigation device 205 to cause the flexible section 230 to bend in one or more directions relative to the rigid section 225. By way of manipulating the flexible section 230 at the end of the rigid section 225, the location of the distal end 220 of the suction/irrigation device 205 inside the patient may be more precisely controlled compared to using the prior art suction/irrigation device 105 of FIG. 1. In embodiments, the rigid section 225 may include a bend 240 that further assists in positioning the distal end 220 at a desired location inside the patient 125 while avoiding interfering contact between the suction/irrigation device 205 and other structures of the robotic system 110. In this manner, the suction/irrigation device 205 provides an improvement over the prior art suction/irrigation device by overcoming the space constraints that make it difficult to locate the distal end of the prior art suction/irrigation device at a desired location inside the patient, thereby providing for more effective and more efficient surgical procedures compared to the prior art suction/irrigation device.

Still referring to FIG. 2A, in embodiments the cannula 210 includes a channel that is in fluid communication with the distal end 220 at one end and suction and irrigation valves that are controlled by a suction switch 245 and irrigation switch 250, respectively, at another end. The suction switch 245 controls (e.g., turns on or off) a suction function of the suction/irrigation device 205 by selectively opening or closing the suction valve. The suction function of the suction/irrigation device 205 provides suction at the distal end 220 via the channel in the cannula 210 and a suction source connected to the suction valve via tubing 260, which may comprise one or more tubes. The irrigation switch 250 controls (e.g., turns on or off) an irrigation function of the suction/irrigation device 205 by selectively opening or closing the irrigation valve. The irrigation function of the suction/irrigation device 205 provides irrigation (e.g., expels liquid, such as water) at the distal end 220 via the channel in the cannula 210 and an irrigation source connected to the irrigation valve via the tubing 260. In this manner, the suction and/or irrigation can be activated and regulated using a valve(s) controlled by a user via switches 245 and 250. The irrigation component may include an activation switch and pump component. The suction and irrigation connections can be made at the back of the housing 215 or anywhere that the design allows for connections of such components. Suction and irrigation tubing, such as tubing 260, may be employed to make these connections at the housing 215.

With continued reference to FIG. 2A, in accordance with aspects of the present disclosure, the cannula 210 includes the flexible section 230 adjacent to the distal end 220. The flexible section 230 may include the distal end 220 or may be interposed between the rigid section 225 and a distal end 220 that is also rigid compared to the flexible section 230. The flexible section 230 allows for selectively bending the cannula 210 at the distal end 220 for steerability of the distal end 220. In embodiments, the flexible section 230 comprises a different material or different configuration than the rigid section 225. In one embodiment, the flexible section 230 is more than half the length of the cannula 210 so that the cannula 210 can be selectively bent along a majority of its length. In one embodiment, the rigid section 225 includes a permanent bend 240 relative to an axial direction of the cannula 210, and the flexible section 230 is between the bend 240 and the distal end 220 (with or without including the tip of the distal end 220).

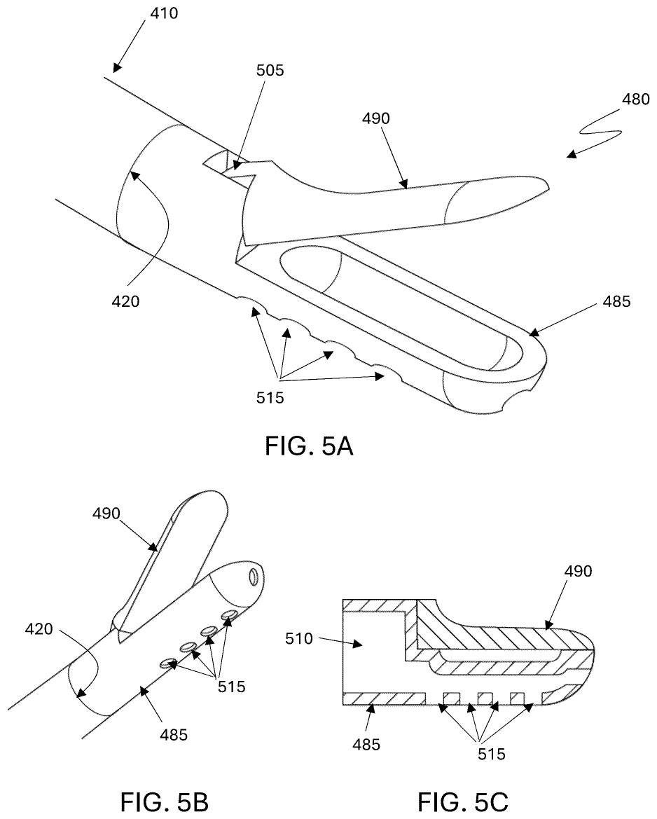

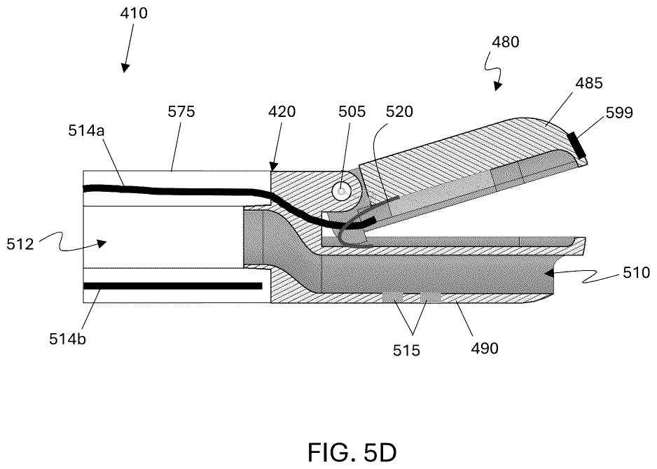

In various embodiments, the suction/irrigation device 205 may further include a grasping actuator 255 that selectively controls (e.g., turns on or off) a grasping function of the suction/irrigation device 205. As discussed herein at FIGS. 5A-C, the distal end 200 may optionally include a grasping component that is selectively actuated by the grasping actuator 255. The grasping actuator 255 may be connected to the grasping component by one or more force transferring cables that are different than the one or more force transferring cables associated with the manipulator 235 for bending the flexible section 230. The grasping actuator 255 may comprise a trigger, slide, wheel, or other actuator that is capable of activating a grasping component as described herein. The grasping component provides the suction/irrigation device 205 with a grasping function of for grasping and manipulating tissue inside the patient.

FIG. 2B shows an embodiment of a suction/irrigation device 205′ that may be used as the suction/irrigation device 205 of FIG. 2A, including a configuration of the cannula 210 in accordance with aspects of the present disclosure. In embodiments, the cannula 210 comprises a rigid component 270 and a flexible component 275. In the configuration shown in FIG. 2B, the proximal end 217 of the cannula 210 is connected to the housing 215. The proximal end 217 of the cannula 210 and the distal end 220 of the cannula 210 are at opposite ends of the cannula along a length of the cannula along an axial direction of the cannula. The flexible component 275 extends from the proximal end 217 of the cannula 210 to the distal end 220 of the cannula 210. The rigid component 270 extends from the proximal end 217 of the cannula 210 to an intermediate location 277 of the cannula 210 along the length of the canula 210 between the proximal end 217 of the cannula 210 and the distal end 220 of the cannula 210. The configuration of the cannula 210 shown in FIG. 2B may be used with any of the embodiments described herein.

In the cannula 210 shown in FIG. 2B, the rigid component 270 comprises a sheath constructed from a relatively rigid material such as metal or rigid plastic. The flexible component 275 comprises a tubular structure constructed from a relatively flexible material that is less rigid than the relatively rigid material of the rigid component 270, such as silicone or plastic that is less rigid than the material of the rigid component. In a preferred embodiment, the flexible component 275 comprises a homogenous and unitary structure that is composed of the same material throughout, such as extruded silicone or plastic, for example.

In the cannula 210 shown in FIG. 2B, the flexible component 275 is inside the rigid component 270 from the proximal end 217 of the cannula 210 to the intermediate location 277 of the cannula 210. In this manner, the rigid section 225 of the cannula is defined from the proximal end 217 of the cannula 210 to the intermediate location 277 of the cannula 210 (e.g., a section where the rigid component 270 forms a sheath around the flexible component 275), and the flexible section 230 of the cannula 210 is defined from the intermediate location 277 of the cannula 210 to the distal end 220 of the cannula 210 (e.g., a section where the flexible component 275 extends outside of the sheath formed by the rigid component 270).

The materials and geometries of the flexible component 275 and the rigid component 270 are selected such that the flexible section 230 of the cannula 210 may be selectively bent in one or more directions along the length of the cannula 210 in response to a user input at the manipulator 235 connected to the housing 215, while the rigid section 225 of the cannula 210 does not bend in response to the same user input at the manipulator 235. In this manner, the flexible section 230 may be selectively bent relative to the rigid section 225 in response to a user input at the manipulator 235.

In accordance with aspects of the present disclosure, the manipulator 235 is configured for selectively controlling bending the flexible section 230 relative to the rigid section 225. In various embodiments, the suction/irrigation device 205 includes one or more force transferring cables connected between the manipulator 235 and the distal end 220. The force transferring cables may extend through one or more guide channels in or attached to the cannula 210. Movement of the manipulator 235 applies tension, compression or torsion force to one or more of the force transferring cables, which causes the flexible section 230 to bend relative to the rigid section 225. The manipulator 235 may comprise a lever, slide, wheel, joystick, etc., that is used to manipulate the bending of the flexible section 230 by changing the tension of one or more of the force transferring cables.

FIG. 2C shows an embodiment of a suction/irrigation device 205″ that may be used as the suction/irrigation device 205 of FIG. 2A. The device 205″ may include the configuration of the cannula 210 shown in FIG. 2B. In this embodiment, the manipulator 235 comprises a joystick connected to an end of the housing 215, the grasping actuator 255 comprises a slide connected to a side of the housing 215, and the suction switch 245 and irrigation switch 250 comprise respective push buttons connected to a side of the housing 215.

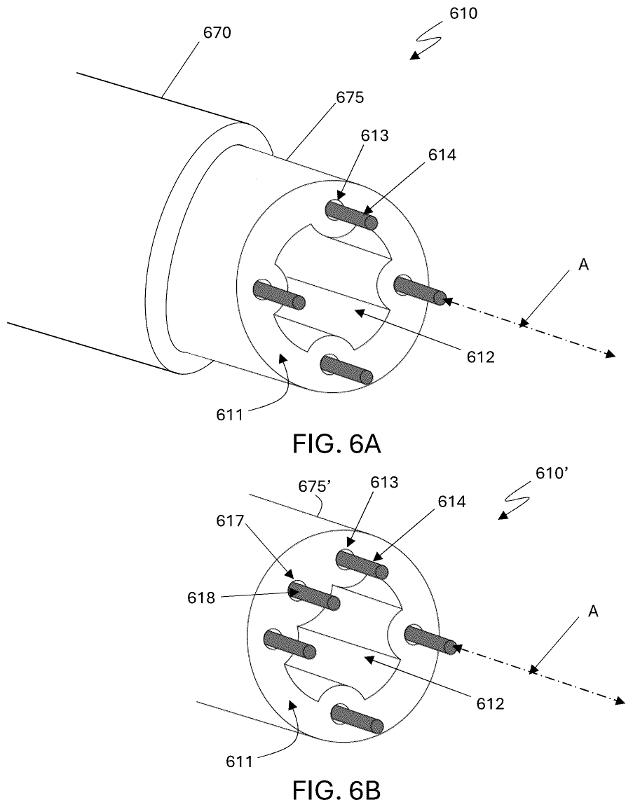

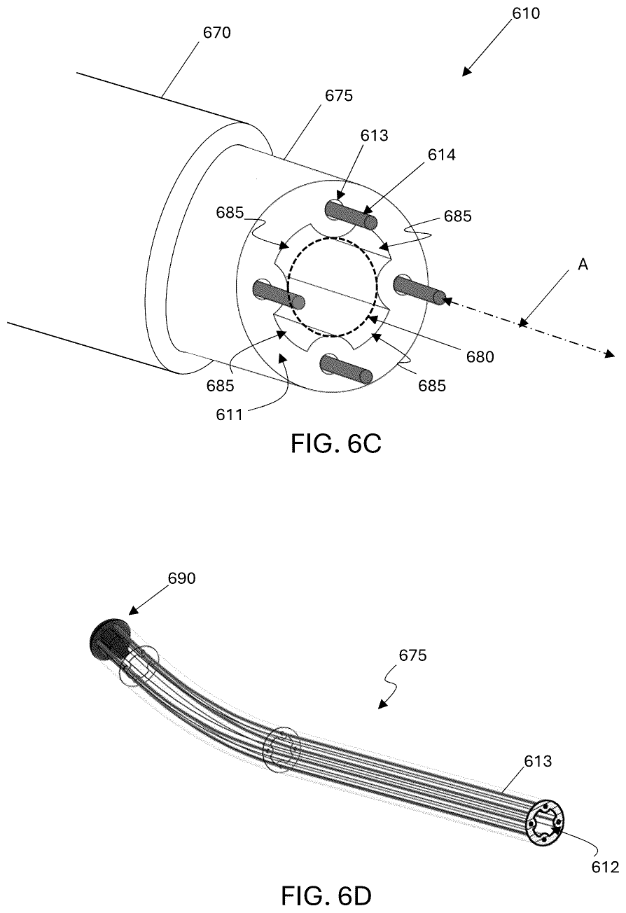

FIG. 2D shows a configuration of the housing 215 that may be used with the suction/irrigation devices 205, 205′, or 205″ in accordance with aspects of the present disclosure. As shown in FIG. 2D, a proximal end 217 of the cannula 210, which includes rigid component 270 and flexible component 275, is connected to the housing 215. In the configuration shown in FIG. 2D, the manipulator 235 comprises a joystick connected to an end of the housing 215 via a gimbal 236 that provides two degrees of freedom and that is attached to force transferring cables 284 that are connected to the gimbal 236 and that extend through an interior of the housing 215 and into passageways formed in the flexible component 275, which may correspond to the passageways 613 shown in FIGS. 6A and 6B and described in more detail herein. As shown in FIG. 2D, the cannula defines a channel 283 (which may correspond to the channel 612 shown in FIGS. 6A and 6B and described in more detail herein) that is configured to convey suction and/or irrigation at the distal end of the cannula. The channel 283 may be in fluidic communication with tubing 260, via an intermediate conduit 281 connected between the flexible component 275 and the tubing 260 (shown in FIG. 2D) or via direct connection between the flexible component 275 and the tubing 260 (not shown). The housing 215 may include a support structure 282 for supporting the intermediate conduit 281, and the support structure 282 may have one or more apertures for the force transferring cables 284 to pass through. In a preferred embodiment, the manipulator 235 is a joystick that allow for two or more degrees of freedom. The joystick may use a ball joint mechanism or a gimbal mechanism 236, to restrain the joystick to the desired number N of degrees of freedom. The force transferring cables 284 are then attached to the joystick at various locations to create the tension and compression in the cables. In embodiments, the force transferring cables 284 (which may correspond to the cables 614 shown in FIGS. 6A and 6B and described in more detail herein) each may comprise a semi-flexible solid core cable that allows for tension and compression forces but remains flexible to bending. In one example, a respective solid Nitinol wire used for each respective one of the force transferring cables 284. In this manner, each force transferring cable 284 is rigid enough to be used in compression when confined to a channel, carry loads under tension, and remain flexible to bending. This improves steering (e.g., bending) of flexible section the cannula by applying tension to one side and compression to the opposite side to offer more precise control and better steering functionality.

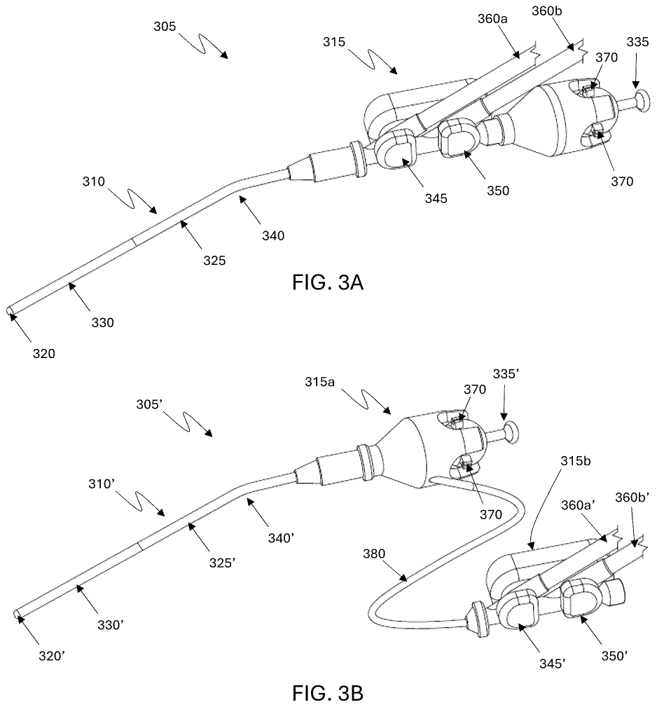

FIG. 3A shows an embodiment of a suction/irrigation device 305 that may be used as the suction/irrigation device 205 of FIG. 2A. In the example shown in FIG. 3A, the suction/irrigation device 305 comprises a cannula 310 and housing 315 corresponding, respectively, to the cannula 210 and housing 215 of FIG. 2A. The cannula 310 comprises a distal end 320, rigid section 325, flexible section 330, and bend 340 corresponding, respectively, to the distal end 220, rigid section 225, flexible section 230, and bend 240 of FIG. 2A. The housing 315 includes a manipulator 335, suction switch 345, and irrigation switch 350 corresponding, respectively, to the manipulator 235, suction switch 245, and irrigation switch 250 of FIG. 2A. Tube 360a fluidically connects a suction valve in the housing 315 to a suction source (e.g., vacuum pump) that is external to the device 305, and tube 360b fluidically connects an irrigation valve in the housing 315 to an irrigation source (e.g., water pump) that is external to the device 305. Each of the suction valve and the irrigation valve in the housing 315 are fluidically connected to a channel inside the cannula 310, where the channel extends from the proximal end of the cannula to the distal end 320 of the cannula. A user may selectively apply suction through the cannula, and via an opening at the distal end 320, by actuating the suction switch 345. A user may selectively apply irrigation through the cannula, and via an opening at the distal end 320, by actuating the irrigation switch 350. A user may selectively bend the flexible section 330 relative to the rigid section 325 by manipulating the manipulator 335. In the example shown in FIG. 3A, the manipulator 335 is a joystick that can move in four different directions to control bending of the flexible section 330 relative to the rigid section 325 in four different directions. In the example shown in FIG. 3A, force transferring cables used in controlling the bending of the flexible section 330 are connected to the manipulator 335 at anchor points 370.

FIG. 3B shows an embodiment of a suction/irrigation device 305′ that may be used as the suction/irrigation device 205 of FIG. 2A. In the example shown in FIG. 3B, the suction/irrigation device 305′ comprises a cannula 310′ and housing corresponding, respectively, to the cannula 210 and housing 215 of FIG. 2A. The cannula 310′ comprises a distal end 320′, rigid section 325′, flexible section 330′, and bend 340′ corresponding, respectively, to the distal end 220, rigid section 225, flexible section 230, and bend 240 of FIG. 2A. The housing includes a first housing section 315a that includes a manipulator 335′ corresponding to the manipulator 235 of FIG. 2A. The housing includes a second housing section 315b that includes a suction switch 345′ and irrigation switch 350′ corresponding, respectively, to the suction switch 245 and irrigation switch 250 of FIG. 2A. Tube 360a′ fluidically connects a suction valve in the second housing section 315b to a suction source (e.g., vacuum pump) that is external to the device 305′, and tube 360b′ fluidically connects an irrigation valve in the second housing section 315b to an irrigation source (e.g., water pump) that is external to the device 305′. Each of the suction valve and the irrigation valve in the second housing section 315b are fluidically connected to a channel inside the cannula 310′ via another one or more tubes 380 that extend between the first housing section 315a and the second housing section 315b, where the channel extends from the proximal end of the cannula to the distal end 320′ of the cannula. A user may selectively apply suction through the cannula, and via an opening at the distal end 320′, by actuating the suction switch 345′. A user may selectively apply irrigation through the cannula, and via an opening at the distal end 320′, by actuating the irrigation switch 350′. A user may selectively bend the flexible section 330′ relative to the rigid section 325′ by manipulating the manipulator 335′. In the example shown in FIG. 3B, the manipulator 335′ is a joystick that can move in four different directions to control bending of the flexible section 330′ relative to the rigid section 325′ in four different directions. In the example shown in FIG. 3B, force transferring cables used in controlling the bending of the flexible section 330′ are connected to the manipulator 335′ at anchor points 370′.

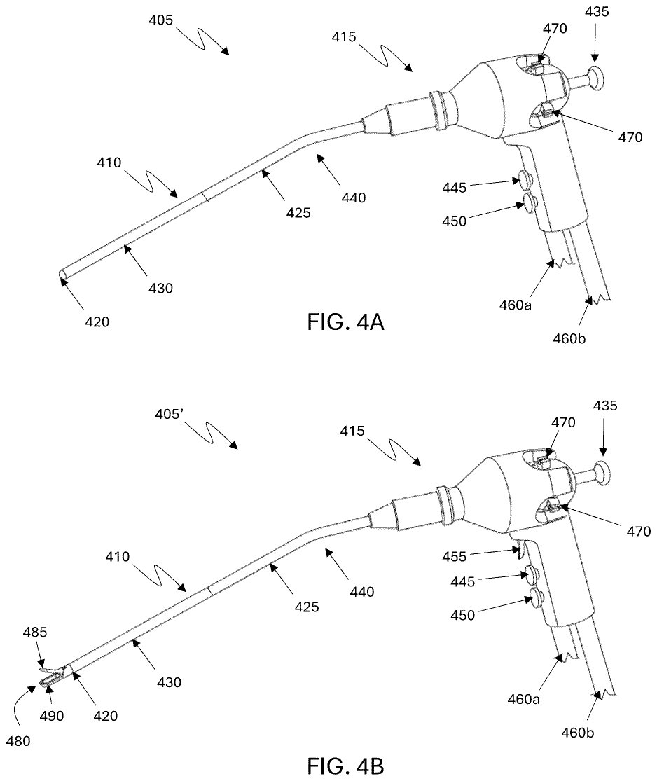

FIG. 4A shows an embodiment of a suction/irrigation device 405 that may be used as the suction/irrigation device 205 of FIG. 2A. In the example shown in FIG. 4A, the suction/irrigation device 405 comprises a cannula 410 and housing 415 corresponding, respectively, to the cannula 210 and housing 215 of FIG. 2A. The cannula 410 comprises a distal end 420, rigid section 425, flexible section 430, and bend 440 corresponding, respectively, to the distal end 220, rigid section 225, flexible section 230, and bend 240 of FIG. 2A. The housing 415 includes a manipulator 435, suction switch 445, and irrigation switch 450 corresponding, respectively, to the manipulator 235, suction switch 245, and irrigation switch 250 of FIG. 2A. As illustrated in FIG. 4A, the housing 415 has a pistol-grip shape that differs from the shape of the housing 315 of FIG. 3A. Tube 460a fluidically connects a suction valve in the housing 415 to a suction source (e.g., vacuum pump) that is external to the device 405, and tube 460b fluidically connects an irrigation valve in the housing 415 to an irrigation source (e.g., water pump) that is external to the device 405. Each of the suction valve and the irrigation valve in the housing 415 are fluidically connected to a channel inside the cannula 410, where the channel extends from the proximal end of the cannula to the distal end 420 of the cannula. A user may selectively apply suction through the cannula, and via an opening at the distal end 420, by actuating the suction switch 445. A user may selectively apply irrigation through the cannula, and via an opening at the distal end 420, by actuating the irrigation switch 450. A user may selectively bend the flexible section 430 relative to the rigid section 425 by manipulating the manipulator 435. In the example shown in FIG. 4A, the manipulator 435 is a joystick that can move in multiple (e.g., four) different directions to control bending of the flexible section 430 relative to the rigid section 425 in multiple (e.g., four) different directions. In the example shown in FIG. 4A, force transferring cables used in controlling the bending of the flexible section 430 are connected to the manipulator 435 at anchor points 470.

FIG. 4B shows an embodiment of a suction/irrigation device 405′ that may be used as the suction/irrigation device 205 of FIG. 2A. The suction/irrigation device 405′ of FIG. 4B has many of the same elements as the suction/irrigation device 405 of FIG. 4A, which are represented by the same reference numbers in both figures. The suction, irrigation, and bending of the flexible section 430 operate the same way in the suction/irrigation device 405′ of FIG. 4B as described with respect to the suction/irrigation device 405 of FIG. 4A. In contrast to the suction/irrigation device 405 of FIG. 4A, the suction/irrigation device 405′ of FIG. 4B includes a grasping element 480 at the distal end 420 and a grasping actuator 455 at the housing 415. In embodiments, the grasping element 480 comprises a first part 485 and a second part 490 that is pivotable relative to the first part 485, where the pivoting of the second part 490 relative to the first part 485 is selectively controlled by a user via the grasping actuator 455. For example, pulling the trigger-shaped grasping actuator 455 may cause the second part 490 to rotate toward the first part 485 in a closing motion (e.g., similar to jaws closing), and releasing the trigger-shaped grasping actuator 455 may cause the second part 490 to rotate away the first part 485 in an opening motion (e.g., similar to jaws opening). The grasping element 480 may be provided with one or more holes in fluidic communication with the channel inside the cannula 410 to convey the suction or irrigation through the grasping element 480, e.g., for application to tissue in the patient.

In various embodiments, the grasping element 480 a bottom jaw and a top jaw. The two jaws may be connected by a joint. This joint may be a pin joint, flexible joint, or any other type of pivot or joint common to those skilled in the art. This joint allows the jaws to open and close the grasper “mouth”. In some embodiments, the bottom jaw is fixed to the end of the steerable suction cannula while the top jaw is connected to the bottom jaw via the joint. In some embodiments, the bottom jaw includes a suction channel that connects the internal suction cannula channel to the end of the grasper. This allows the grasper tip to still allow for suction functionality. This suction channel may include suction relief holes along the side to keep the main suction channel from being occluded. This suction channel may be a unique shape that allows for the cross section of the suction channel not to be reduced. In some embodiments, the suction channel maintains its cross section as it changes shape and transitions through the grasper

FIGS. 5A, 5B, 5C, and 5D show aspects of an exemplary grasping element 480 as shown in FIG. 4B in accordance with aspects of the present disclosure. In embodiments, the grasping element 480 includes a first part 485 and a second part 490 that is pivotable relative to the first part 485. Either of the parts 485 and 490 may be provided with ridges, teeth, roughening, or sharp surfaces to assist in grasping tissue inside the patient. As shown in FIG. 5A, the second part 490 may be pivotally connected to the first part 485 at a hinge 505. The hinge 505 may comprise a pin the defines the pivot axis, a living hinge, or any other suitable type of hinge connection that permits rotational movement of the second part 490 relative to the first part 485. As shown in FIG. 5D, the hinge 505 may include a bias element 520 that urges the second part 490 away from the first part 485, so that the grasping element 480 automatically opens when the user releases the grasping actuator. The bias element may comprise a spring that contacts the second part 490 and the first part 485, or a resiliently flexible material that is integrally formed with one or both of the second part 490 and the first part 485. In embodiments, and as shown in FIGS. 5A, 5B, and 5D, the grasping element 480 is connected to the cannula 410 at the distal end 420 of the cannula 410. The cannula 410 may include a rigid component (not shown) and a flexible components 575 corresponding respectively to the rigid component 270 and the flexible components 275 shown in FIG. 2B. The flexible component 575 of FIG. 5D may have a configuration shown in FIG. 6B and may define a channel 512 that corresponds to the channel 612 shown in FIG. 6B. A force transferring cable 514a, which may correspond to force transferring cable 618 of FIG. 6B, may run through the flexible component 575 and be connected to the grasping element 480 for moving the first part 485 relative to the second part 490. One or more force transferring cables 514b, which may correspond to one or more of cables 614 in FIG. 6B, may run through the flexible component 575 and terminate within the flexible component 575 for providing bending of the flexible component 575 as described herein.

In embodiments, and as shown in FIGS. 5B-5D, the grasping element 480 includes a channel 510 that is in fluidic communication with the channel 512 in the cannula 410 when the grasping element 480 is connected to the distal end 420. In embodiments, and as shown in FIGS. 5A-C, the grasping element 480 includes one or more ports 515 in fluidic communication with the channel 510 and the exterior of the grasping element 480, so that the grasping element 480 conveys suction or irrigation from the channel inside the cannula 410 to the exterior of the grasping element 480.

The grasping element 480 shown in FIGS. 5A-C is exemplary and not limiting, and other shapes and configurations of grasping element may be used with the suction/irrigation device 405′ of FIG. 4B. Additionally, although not shown, each of the suction/irrigation device 305 of FIG. 3A and the suction/irrigation device 305′ of FIG. 3B may be modified to include as grasping actuator (e.g., similar to grasping actuator 455 of FIG. 4B) and a grasping element (e.g., similar to grasping element 480 of FIG. 4B).

With continued reference to FIGS. 5A-D, in an embodiment, the top jaw (e.g., first part 485) is used as the movable jaw to create an open and closing mouth section in the grasper. This opening and closing action can be controlled by tension or compression cables similar to the cables used for the steering mechanism. A tension/compression cable is put into tension or compression via an actuation device (such as a trigger or slider) in the handle and causes the upper jaw to close down on the bottom jaw (e.g., second first part 490). The jaws can be returned open via a spring (such as a torsion spring), built in bias towards the open position or by using the cable as a compression/force transferring cable to return the jaw open when a force is applied to the cable.

FIG. 6A-6C show aspects of a suction/irrigation device usable in single-port robotic surgery in accordance with aspects of the present disclosure. FIG. 6A shows a sectional view of a cannula 610 that may be used in any of the devices 205, 205′, 205″, 305, 305′, 405, and 405′ as described herein. In embodiments, the cannula 610 a rigid component 670 (corresponding to rigid component 270 of FIG. 2C) and a flexible component 675 (corresponding to flexible component 275 of FIG. 2C), and the flexible component 675 includes a cylindrical body 611 (e.g., a tube) that defines the channel 612 that is configured to fluidically communicate suction or irrigation between the respective valves in the housing and the distal end of the cannula, as described herein. In embodiments, and as shown in FIG. 6A, the flexible component 675 passageways 613 that accommodate force transferring cables 614. In embodiments, the passageways 613 are separate from and not in fluidic communication with the channel 612. For example, the passageways 613 may be formed in a material of the flexible component 675 and are separated from the channel 612 by the material of the flexible component 675. In embodiments, the force transferring cables 614 may move axially along a length direction of the passageways 613, e.g., as represented by arrow A in FIG. 6A, in response to user input at the manipulator such as manipulator 335 of FIG. 3A. In embodiments, each of the force transferring cables 614 has one end connected to the manipulator and another end anchored in the flexible section of the canula.

FIG. 6A shows four passageways 613 and four force transferring cables 614 (only one set of the four being numbered) for use with a manipulator that is designed to move in four directions, such as manipulator 335 of FIG. 3A. However, implementations may utilize a different manipulator that has a different number of movement directions and is connected to the flexible section via a different number of force transferring cables. In one example, the manipulator is actuatable in a single direction and is connected to the flexible section via one force transferring cable such that the manipulator may be used to bend the flexible section relative to the rigid section in one direction. This arrangement provides unidirectional steerability of the cannula tip and allows for steering of the cannula tip in a single direction while rotating the cannula along its axis for additional manipulation. In another example, the manipulator is actuatable in two different directions and is connected to the flexible section via two force transferring cables, such that the manipulator may be used to bend the flexible section relative to the rigid section in two different directions. This arrangement provides bidirectional steerability in the cannula tip and allows for steering of the cannula tip in two directions while rotating the cannula along its axis for additional manipulation. In another example, the manipulator is actuatable in four different directions and is connected to the flexible section via three or four force transferring cables, such that the manipulator may be used to bend the flexible section relative to the rigid section in all directions. This arrangement provides multidirectional steerability in the cannula tip and allows for steering of the cannula tip in multiple directions

FIG. 6B shows a sectional view of an exemplary cannula 610′ that may be used in any of the devices described herein that utilize a grasping actuator and grasping element. FIG. 6B shows the flexible component 675′ but omits the rigid component for clarity, although it is understood that the cannula 610′ may include a rigid component similar to the rigid component 670 shown n FIG. 6A. The flexible component 675′ of FIG. 6B includes the cylindrical body 611, channel 612, one or more passageways 613, and one or more force transferring cables 614 that function in the same manner as described with respect to FIG. 6A. The flexible component 675′ of FIG. 6B additionally includes a passageway 617 that accommodates a grasping force transferring cable 618 that is connected between the grasping actuator (e.g., grasping actuator 455) and grasping element (e.g., grasping element 480). In embodiments, the passageway 617 and grasping force transferring cable 618 are separate from the one or more passageways 613, and one or more force transferring cables 614. In embodiments, the grasping force transferring cable 618 is configured to actuate the grasping element in response to the user providing input at the grasping actuator.

FIG. 6C shows the cannula 610 of FIG. 6A and depicts an example of how the flexible component is shaped to form a main area of the channel and one or more auxiliary areas of the channel in accordance with aspects of the present disclosure. As shown in FIG. 6C, an exterior surface of the flexible component 675 has a first shape in cross section, and an interior surface of the flexible component 675 has a second shape in the same cross section, with the second shape being different than the first shape. The first shape may be circular, oval, elliptical, or polygonal, for example. The second shape defines a main area 680 of the channel and one or more auxiliary areas 685 of the channel. In a preferred embodiment, the main area 680 of the channel is substantially centrally located within the flexible component 675, and the one or more auxiliary areas 685 of the channel are at a perimeter of the main area 680 of the channel and in fluidic communication with the main area 680 of the channel. The second shape of the flexible component 675 may include one or more protrusions (e.g., surfaces that extend inward toward a center of the flexible component when viewed in cross section), wherein the one or more protrusions define the main area 680 and the one or more auxiliary areas 685 of the channel. In a preferred embodiment, the one or more protrusions are at locations where the one or more cables 614 are embedded in and run through the flexible component 675 in the axial direction of the flexible component along the length of the flexible component 675. Advantageously, the one or more auxiliary areas of the channel provide for continued suction and/or irrigation in the channel in the event that the main area of the channel becomes occluded, e.g., by tissue, blood clot, etc. FIG. 6D shows a transparent view of an exemplary configuration of the flexible component 675 of FIGS. 6A and 6C showing the channel 612 and the cables 614 running through the material of the flexible component 675 to a distal end 690 of the cannula.

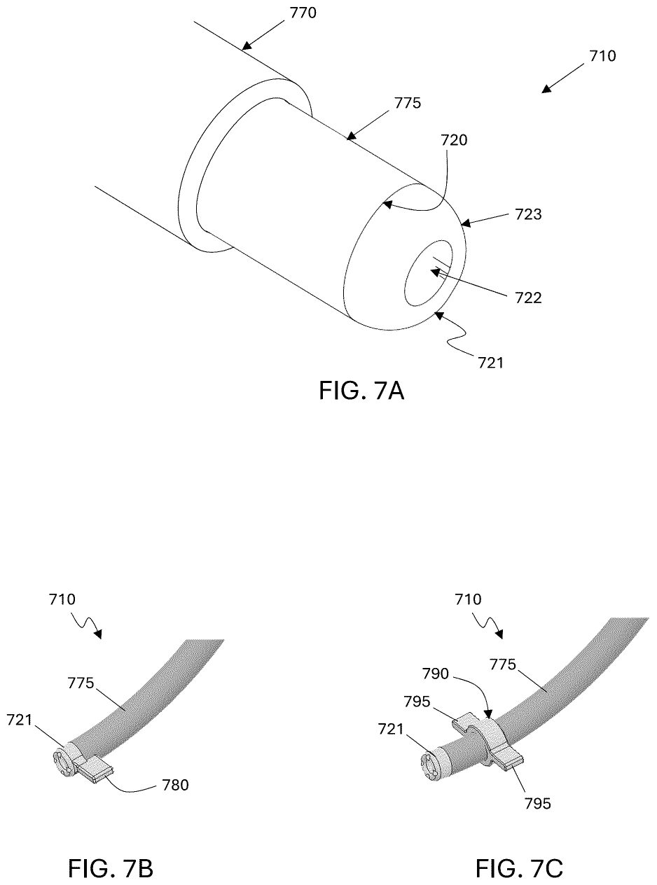

FIG. 7A shows a view of an exemplary cannula 710 that may be used in any of the devices 205, 205′, 205″, 305, 305′, 405, 405′ as described herein. The cannula 710 includes a distal end 720 which may correspond to the distal ends 220, 320, 320′, and 420. In embodiments, a blunt tip 721 is connected to the distal end 720. The cannula 710 may have the configuration of the canula 210 of FIG. 2B comprising a rigid component 770 (corresponding to rigid component 270 of FIG. 2C) and a flexible component 775 (corresponding to flexible component 275 of FIG. 2C). In embodiments, the tip 721 includes a channel 722 that is in fluidic communication with the channel in the cannula 710 when the tip 721 is connected to the distal end 720. In this manner, the tip 721 including the channel 722 conveys suction or irrigation from the channel inside the cannula 710 to the exterior of the tip 721. In embodiments, the tip 721 includes a rounded profile 723 at its distal end that reduces trauma to the tissue of the patient. In embodiments, the tip 721 is connected to the distal end 720 or is integrally formed with the distal end 720. In embodiments, a cannula having a tip such as tip 721 may be used when a grasping element is not desired.

FIG. 7B shows a configuration of the cannula 710 comprising the flexible component 775, rigid component (not shown), and tip 721 at the distal end. In this configuration, the tip 721 includes one or more protrusions 780, which may be in the form of tabs that extend radially outward from the tubular shape of the tip 721. In embodiments, the protrusions 780 are configured to be grabbed by one of the robotic instruments 120 of the robotic system 110 of FIG. 1 (or other robotic system), so that the robotic instrument 120 may be used to apply a force to the cannula 710 for bending the flexible section of the cannula, e.g., for positioning the distal end of the cannula in the working space.

FIG. 7C shows a configuration of the cannula 710 comprising the flexible component 775, rigid component (not shown), and tip 721 at the distal end. In this configuration, a clip 790 that includes one or more protrusions 795 is connected to the flexible component 775. The protrusions 795 may be in the form of tabs that extend radially outward from the tubular shape of the flexible component 775. In embodiments, the protrusions 795 are configured to be grabbed by one of the robotic instruments 120 of the robotic system 110 of FIG. 1 (or other robotic system), so that the robotic instrument 120 may be used to apply a force to the cannula 710 for bending the flexible section of the cannula, e.g., for positioning the distal end of the cannula in the working space. The clip 790 may be used with embodiments that include the blunt tip or the grasping element.

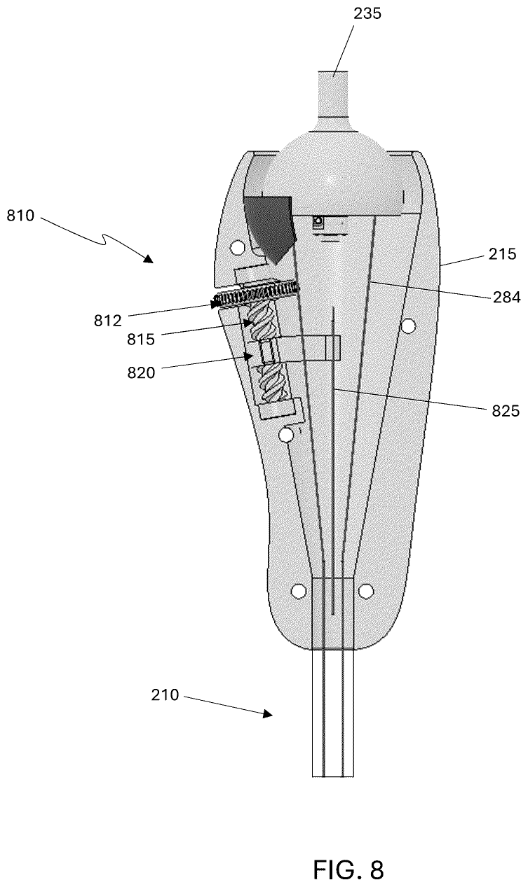

FIG. 8 shows a configuration of a grasping actuator that that may be used with a cannula in accordance with aspects of the present disclosure. Several actuation mechanisms can be used to apply tension and compression to the force transferring cable of the grasper. These can include triggers, buttons, sliders, etc. A preferred embodiment utilizes a linear screw actuation mechanism 810 that uses the turning of the screw 815 to move a slider 820 that applies tension and compression to the cable 825 that is connected to the grasping element 480 (e.g., grasping element 480) at the distal end of the cannula (e.g., cannula 210). A wheel 812 (e.g., a thumb wheel) may be fixed to the screw 815 for a user to turn the screw 815. Cable 825 may correspond to cable 618 of FIG. 6B. The linear screw actuation mechanism 810 can be controlled via a wheel trigger mechanism. The linear screw actuation mechanism 810 may be incorporated in the housing 215, e.g., in the manner shown in FIG. 8 or in any other suitable manner and may be used as the grasping actuator 255 in various embodiments. Alternatively to the wheel 812, the linear screw actuation mechanism 810 may utilize a trigger mechanism with a rack gear attached to a trigger and a pinion gear attached to the screw 815. When the trigger is pressed it translates that motion into the screw 815 via the rack and pinion gear mechanism and converts it to a rotation. This rotation then converts to linear motion again at the slider 820. The cable can also be used to transmit the torsion and rotation through the channel of the steering cannula to the grasper to actuate the gripping mechanism.

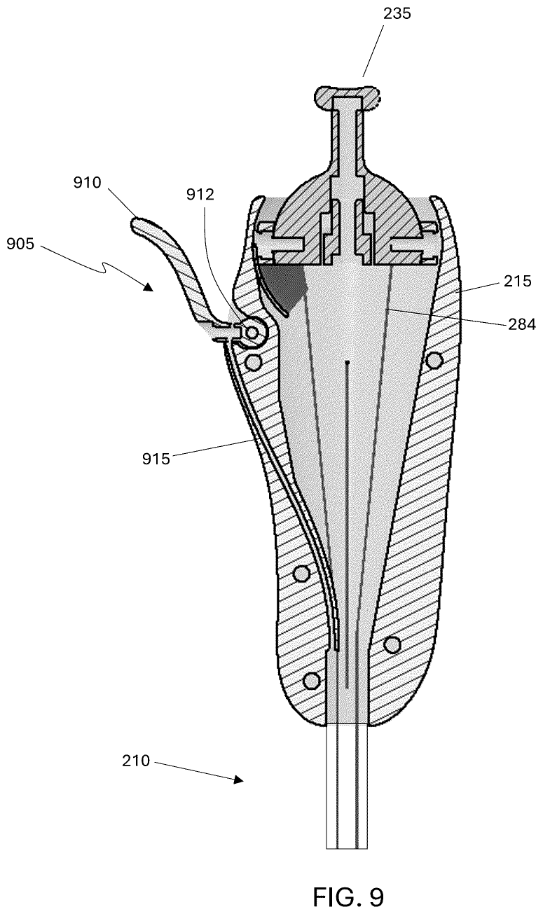

FIG. 9 shows a configuration of a grasping actuator that may be used with a cannula in accordance with aspects of the present disclosure. A trigger mechanism 910 with a pivot joint 912 on the housing 215 can also be used to apply tension and compression to a cable 925 within a cable channel 915. The cable within the cable channel 915 may be connected at one end to the trigger 905 and at its opposite end to the grasping element 480 (e.g., grasping element 480) at the distal end of the cannula (e.g., cannula 210) and may correspond to cable 618 of FIG. 6B. The housing 215 may include a cable channel.

Any embodiment of the suction/irrigation device described herein (e.g., 205, 205′, 205″, 305, 305′, 405, 405′) may include a magnetic component on the suction tip or grasping element 480 to allow for the use of the magnetic tip for activities such as attaching a suture. An example is magnet 599 attached to the grasping element 480 in FIG. 5D.

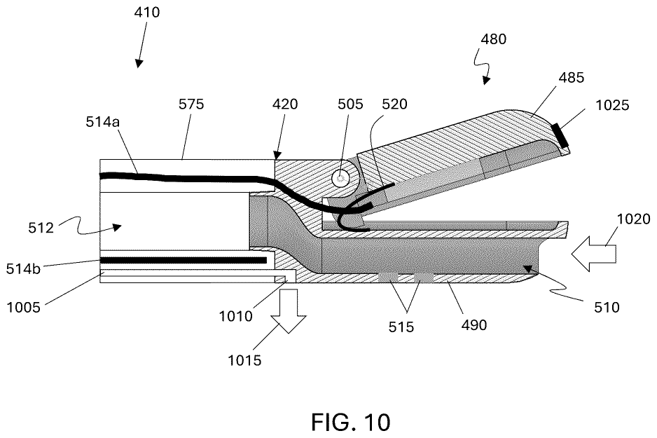

Any embodiment of the suction/irrigation device described herein (e.g., 205, 205′, 205″, 305, 305′, 405, 405′) may include a pressurization system. Pressurization systems are often used to maintain an open cavity to work within (e.g., inside the body of the patient). Often a suction component may alter this pressure. In embodiments, the suction/irrigation device may have a gas flow inlet component that matches the flow outlet via suction to maintain consistent pressure. Therefore, this would create a closed loop suction and pressurization system that maintains pressure in the working cavity (e.g., inside the body of the patient) during all use cases of the device. An example is shown in FIG. 10, in which a separate channel 1005 in the flexible component 575 of the cannula 410 is employed to allow pressurized air to flow in through the steerable cannula. In embodiments, this channel 1005 may be fluidically connected to a corresponding channel 1010 in the grasping element 480 thereby allowing pressurized gas 1015 to be flowed into the working cavity (e.g., inside the body of the patient). In this embodiment, the channel 513 in the cannula 410 and the channel 510 in grasping element 480 provide for suction indicated by arrow 1020. In this manner, the inventive system allows pressurized air to flow in and suction to remove air simultaneously. These flowrates can be matched to maintain pressure. Another embodiment can use a separate inflow location separate from the steerable suction. Another embodiment may use a single channel to switch between suction, pressurization and/or irrigation via bi-directional valves. A further embodiment includes the use of a pressure sensor at the tip of the steerable cannula or grasper, e.g., as shown at pressure sensor 1025 in FIG. 10. This pressure sensor can be used to monitor and adjust the pressure in the working cavity (e.g., inside the body of the patient). This would also be used to adjust the flowrates of the both suction and pressurization components together or independently. Additional sensors such as flowrate, etc., may be employed to create a feedback control system for the pressurization, suction and irrigation systems. Furthermore, control parameters such as over-pressurization and intra-cavity pressure deviations can be monitored and controlled.

The pressurization system may use air or any other gases suitable for surgical use. Another embodiment allows for the conditioning of the gas. Examples of conditioning may include humidification, temperature control, smoke filtration, etc.

Additional aspects of the invention include manufacturing and/or using a suction/irrigation device described herein. Even further aspects of the invention include providing instructions for using a suction/irrigation device described herein. The instructions may be at least one of printed and video.

It is noted that the foregoing examples have been provided merely for the purpose of explanation and are in no way to be construed as limiting of implementations of the present invention. While aspects of the present invention have been described with reference to an exemplary embodiment, it is understood that the words which have been used herein are words of description and illustration, rather than words of limitation. Changes may be made, within the purview of the appended claims, as presently stated and as amended, without departing from the scope and spirit of the present disclosure in its aspects. Although implementations of the present invention have been described herein with reference to particular means, materials and embodiments, implementations of the present invention are not intended to be limited to the particulars disclosed herein; rather, implementations of the present invention extend to all functionally equivalent structures, methods and uses, such as are within the scope of the appended claims.

What is claimed:

1. A medical device, comprising:
a cannula having a distal end and a proximal end opposite the distal end;
a housing connected to the proximal end of the cannula; and
a manipulator connected to the housing;
wherein the cannula includes a rigid component that extends from the proximal end of the cannula to an intermediate location between the proximal end of the cannula and the distal end of the cannula;
the cannula comprises a flexible component that extends from the proximal end of the cannula to the distal end of the cannula;
the cannula includes a rigid section in which the flexible component is inside the rigid component and a flexible section in which the flexible component is outside the rigid component;
the manipulator is connected to the flexible component in a manner that permits selective bending of the flexible section relative to the rigid section in response to user input at the manipulator; and
the flexible component includes a channel that is configured to convey suction and/or irrigation at the distal end of the cannula.

2. The medical device of claim 1, further comprising:
a suction valve; and
a suction switch that selectively opens and closes the suction valve to provide suction at the distal end of the cannula.

3. The medical device of claim 1, further comprising:
an irrigation valve; and
an irrigation switch that selectively opens and closes the irrigation valve to provide irrigation at the distal end of the cannula.

4. The medical device of claim 1, further comprising:
a suction valve;
a suction switch that selectively opens and closes the suction valve to provide suction at the distal end of the cannula;
an irrigation valve; and
an irrigation switch that selectively opens and closes the irrigation valve to provide irrigation at the distal end of the cannula.

5. The medical device of claim 1, further comprising a permanent bend in the rigid component along an axial direction of the cannula.

6. The medical device of claim 1, further comprising a blunt tip connected to the flexible component at the distal end of the cannula, wherein the blunt tip comprises:
a tip channel that conveys suction or irrigation from the channel inside the flexible component to an exterior of the blunt tip; and
a rounded profile at a distal end of the blunt tip.

7. The medical device of claim 1, further comprising:
a grasping element connected to the flexible component at the distal end of the cannula; and
a grasping actuator that is configured to selectively activate the grasping element in response to user input at the grasping actuator.

8. The medical device of claim 7, wherein the grasping element comprises a first part and a second part that is pivotable relative to the first part, wherein the pivoting of the second part relative to the first part is selectively controlled by a user input at the grasping actuator.

9. The medical device of claim 7, wherein the grasping element comprises a grasping element channel in fluidic communication with the channel in the cannula, and one or more ports in fluidic communication with the grasping element channel and an exterior of the grasping element, such that the grasping element conveys suction or irrigation from the channel inside the cannula to the exterior of the grasping element.

10. The medical device of claim 7, wherein the grasping element is connected to the grasping actuator by a cable that runs in a passageway defined by the cannula.

11. The medical device of claim 7, wherein the grasping actuator comprises a screw and slider.

12. The medical device of claim 1, wherein the flexible section extends from the intermediate location to the distal end of the cannula.

13. The medical device of claim 1, wherein the flexible component comprises a homogenous and unitary structure.

14. The medical device of claim 1, wherein:
an exterior surface of the flexible component has a first shape in cross section;
an interior surface of the flexible component has a second shape in the same cross section, with the second shape being different than the first shape; and
the second shape includes a main area of the channel and one or more auxiliary areas of the channel.

15. The medical device of claim 14, wherein the second shape includes one or more protrusions that define the main area of the channel and the one or more auxiliary areas of the channel.

16. The medical device of claim 15, wherein the one or more protrusions are at locations where one or more cables are embedded in and run through passageways in the flexible component in an axial direction of the flexible component along a length of the flexible component.

17. The medical device of claim 1, wherein the manipulator is connected to the flexible section of the cannula by one or more cables that run in one or more passageways defined by the flexible component.

18. The medical device of claim 17, wherein the one or more passageways are in a material of the flexible component and are separated from the channel by the material of the flexible component.

19. The medical device of claim 17, wherein the one or more passageways include:
a first passageway that accommodates a first cable that is operatively connected between a grasping actuator at the housing and a grasping element connected to the flexible component at the distal end of the cannula; and
a second passageway that accommodates a second cable having a first end connected to the manipulator and a second end anchored in the flexible component of the cannula.

20. The medical device of claim 17, wherein the manipulator is actuatable in four different directions and is connected to the flexible section via four cables of the one or more cable such that the manipulator may be used to bend the flexible section relative to the rigid section in four different directions.

21. The medical device of claim 1, wherein the medical device is configured to be used in single-port surgery.

22. The medical device of claim 1, further comprising a pressurization system.

23. The medical device of claim 22, wherein the pressurization system comprises a pressurization channel in the flexible component that is configured to convey pressurized gas to the distal end of the cannula.

24. The medical device of claim 23, wherein the pressurization channel is separate from the channel such that pressurized gas may be conveyed via the pressurization channel simultaneously with suction being conveyed via the channel.

25. The medical device of claim 22, wherein the pressurization system comprises a pressure sensor at the distal end of the cannula or at a grasping element connected to the distal end of the cannula.

26. The medical device of claim 1, further comprising a magnet at the distal end of the cannula or at a grasping element connected to the distal end of the cannula.

27. The medical device of claim 1, further comprising a tab extending radially outward from the flexible component or from a blunt tip connected to the flexible component at the distal end of the cannula.
